(19) United States
(12) Patent Application Publication
(10) Pub. No.: US 2017/0203130 A1
(43) Pub. Date: Jul. 20, 2017

(54) SHOCKWAVE HEAD STRUCTURE HAVING BENDING ANGLE

(71) Applicant: Lite-Med Inc., Taipei (TW)

(72) Inventors: Po-Hsiang KUO, Taipei (TW); Yung-Chen SU, Taipei (TW); Chia-Chi LIN, Taipei (TW)

(73) Assignee: Lite-Med Inc.

(21) Appl. No.: 15/071,531

(22) Filed: Mar. 16, 2016

(30) Foreign Application Priority Data
Jan. 14, 2016 (TW) 105101131

(51) Int. Cl. A61N 7/00

(52) U.S. Cl. CPC A61N 7/00

(57) ABSTRACT

The present invention discloses a shockwave head structure having a bending angle. The shockwave head structure includes a shockwave waveguide body and a path regulation unit. The path regulation unit is located inside the shockwave waveguide body and can refract or reflect an energy wave entering from an incident port of the shockwave waveguide body to an exit port of the shockwave waveguide body, thereby regulating the path of the energy wave. With the implementation of the present invention, the shockwave head structure can be applied to different body parts or body positions. Thus, the practicability of the shockwave head structure is increased.

### BACKGROUND OF THE INVENTION

1. Technical Field

The present invention relates to a shockwave head structure having a bending angle, and particularly relates to a shockwave head structure used in ultrasound care and medical devices.

2. Description of Related Art

The history of using ultrasound in the field of medical treatment is not that long. Ultrasound has mainly started to be used in medicine in the 20th century. However, today, it has an irreplaceable position in modern medical diagnostics. Nowadays, ultrasonography is widely used in medical treatment. Besides its use in diagnosis, it also has a guiding function in therapeutic processes.

For example, medical ultrasonography is commonly used in the fields of cardiology, endocrinology, gastroenterology, gynecology, obstetrics, ophthalmology, urology, cardiovasculogy, ultrasound imaging, ophthalmology etc. In addition to its use in medical treatment, it also has numerous other applications in health care and aesthetic-related areas such as ultrashape, which mainly uses ultrasound precision to annihilate stubborn fats under the skin for people who don't want surgery, but want local body shaping. It has advantages such as being painless, not leaving wounds/scars, not using anaesthesia, and not having a recovery phase.

The easy-to-use ultrasound equipment is for instance a hand-held probe, which is often referred to as a probe and principally used by being placed on a patient's body and making a scanning movement. The prior art hand-held probes have the following shockwave conducting directions: 1, Focus mode: This mode is designed according to the oval concave lens theory, in which, after the shockwave is emitted from the focal point of the oval concave lens and reflected from the mirror surface, it is focused again at another focal point of the oval concave lens; 2. Radiation mode: In this mode, the shockwave is dispersed around in all directions from the “source”; 3. Plane mode: In this shockwave source design, only the shockwaves that are in parallel with the narrow exit port can be emitted out through the exit port, and therefore it is restricted a certain plane direction.

The above mentioned various shockwave sources are all located at the same straight line with their shockwave exit ports, and therefore, during use, if the shockwave is required to be directed towards a different body position or required to be emitted towards a different direction, it is usually encountered with great restrictions, which causes serious difficulty for the shockwaves to accurately pass through the skin and the tissues in the body, which also substantially affects its efficiency of use.

### SUMMARY OF THE INVENTION

The present invention relates to a shockwave head structure having a bending angle, primarily solving problem of the prior art shockwave head structures, which cannot easily regulate the emission angle of shockwaves, and thus cause inconvenience during use.

The present invention provides a shockwave head structure having a bending angle, comprising: a shockwave waveguide body comprising an incident port and an exit port; and a path regulation unit positioned inside the shockwave waveguide body in order to refract or reflect an energy wave entering from the incident port to the exit port.

Implementation of the present invention at least involves the following inventive steps:

1. It can be directed towards different body positions or operated towards different directions, so that the receivers of the shockwaves do not have to change their body positions or go into difficult postures.

2. It enables the shockwaves to accurately pass through the skin and the tissues within the body so as to achieve a suitable effect.

3. It can improve the therapy conditions or expand the range that can be treated.

The features and advantages of the present invention are detailed hereinafter with reference to the preferred embodiments. The detailed description is intended to enable a person skilled in the art to gain insight into the technical contents disclosed herein and implement the present invention accordingly. In particular, a person skilled in the art can easily understand the objects and advantages of the present invention by referring to the disclosure of the specification, the claims, and the accompanying drawings.

### BRIEF DESCRIPTION OF THE SEVERAL VIEWS OF THE DRAWINGS

FIG. 1 is a shockwave head structure having a bending angle in an embodiment of the present invention;

FIG. 2 is a shockwave head structure having a bending angle in an embodiment of the present invention, wherein a reflecting unit serves as a path regulation unit; and

FIG. 3 is a shockwave head structure having a bending angle in an embodiment of the present invention, wherein a refracting unit serves as a path regulation unit.

### DETAILED DESCRIPTION OF THE EMBODIMENTS OF THE INVENTION

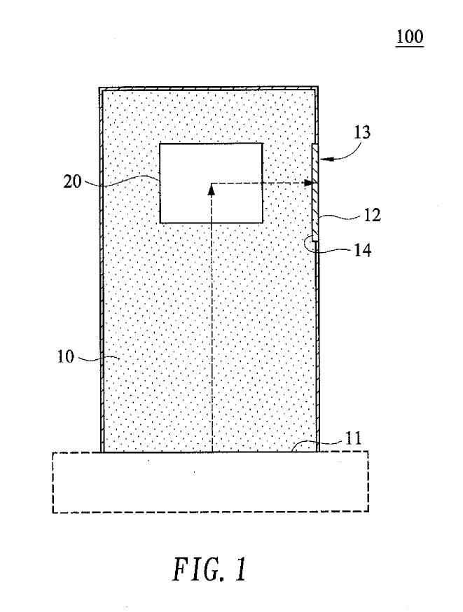

Referring to FIG. 1, a shockwave head structure has a bending angle 100, comprising: a shockwave waveguide body 10; and a path regulation unit 20.

The shockwave waveguide body 10 is mainly used for providing shockwave or an ultrasound energy transfer medium. The shockwave waveguide body 10 comprises an incident port 11 and an exit port 12, and the exit port 12 is not positioned along the straight path corresponding to the incident port 11. In other words, the incident port 11 and the exit port 12 are not positioned at the two opposite ends of the shockwave waveguide body 10. For instance, if the incident port 11 is positioned at the bottom of the shockwave waveguide body 10, then the exit port 12 is found at the lateral side of the shockwave waveguide body 10. The shockwave waveguide body 10 can be made of a glass, a ceramic, a stainless steel, a high density polyethylene, or an acrylic material. The energy wave can be shockwave or ultrasound wave.

In order to ensure effective ejection of energy waves out of the shockwave waveguide body 10 or conveniently filling various media into the shockwave waveguide body 10, the exit port 12 of the shockwave waveguide body 10 has an opening 13, and the opening 13 is sealed by a sealing membrane 14.

The path regulation unit 20 is located inside the shockwave waveguide body 10. Refracting or reflecting functions can be generated by means of the path regulation unit 20, wherein an energy wave entering from an incident port 11 of the shockwave waveguide body 10 is refracted or reflected to an exit port 12 of the shockwave waveguide body 10.

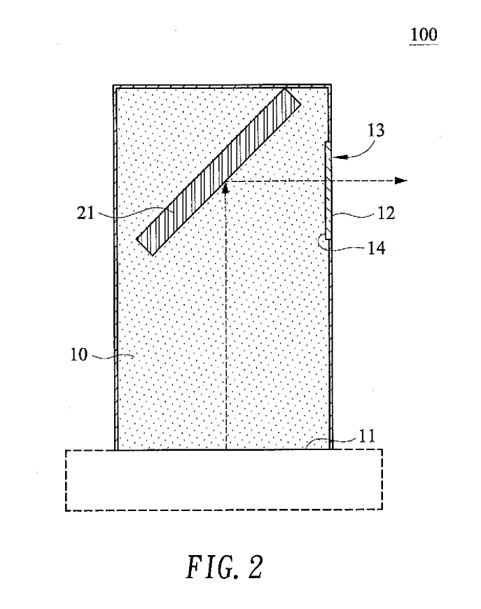

Referring to FIG. 2, when the path regulation unit 20 is a reflecting unit 21, the path regulation unit 20 comprises a reflecting plane positioned on the path of the energy wave, so that it can effectively reflect the energy wave towards the exit port 12. The shockwave waveguide body 10 is filled with an acoustic medium, such as water, and the reflecting unit 21 can be made of a stainless steel, a high density polyethylene, or an acrylic material.

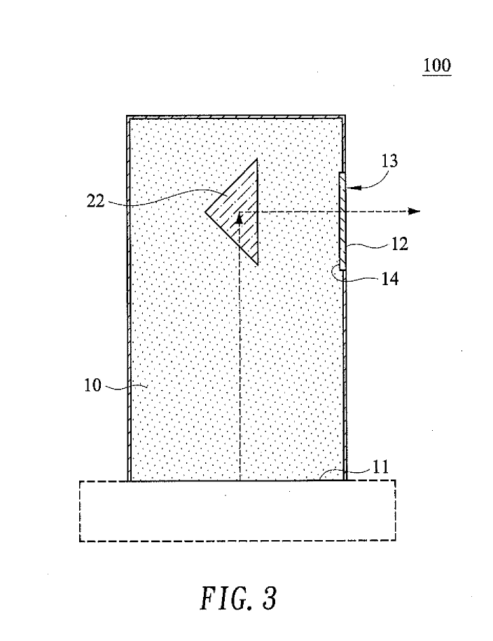

Referring to FIG. 3, when the path regulation unit 20 is a refracting unit 22, the refracting unit 22 is positioned on the path of the energy wave, so that it can effectively perform refraction on the energy wave in order to regulate the travelling direction of the energy wave. Also, the shockwave waveguide body 10 can be filled with an acoustic medium, such as a water medium, a high density polyethylene medium, or a silica gel, and the refracting unit 22 can be made of a water sack, a high density polyethylene, or a silica gel material.

The foregoing embodiments are provided to illustrate and disclose the technical features of the present invention so as to enable persons skilled in the art to understand the disclosure of the present invention and implement the present invention accordingly, and are not intended to be restrictive of the scope of patent protection given to the present invention. Hence, all equivalent modifications and variations made to the foregoing embodiments without departing from the spirit embodied in the disclosure of the present invention should fall within the scope of the present invention as set forth in the appended claims.

What is claimed is:

1. A shockwave head structure having a bending angle, comprising:
a shockwave waveguide body comprising an incident port and an exit port; and
a path regulation unit positioned inside the shockwave waveguide body in order to refract or reflect an energy wave entering from the incident port to the exit port.

2. The shockwave head structure of claim 1, wherein the shockwave waveguide body is made of a glass, a ceramic, a stainless steel, a high density polyethylene, or an acrylic material.

3. The shockwave head structure of claim 1, wherein the shockwave waveguide body has an opening formed at the exit port, and the opening is sealed by a sealing membrane.

4. The shockwave head structure of claim 1, wherein the path regulation unit is a reflecting unit, which has a reflecting plane positioned on the path of the energy wave.

5. The shockwave head structure of claim 4, wherein the shockwave waveguide body is filled with an acoustic medium.

6. The shockwave head structure of claim 4, wherein the reflecting unit is made of a stainless, a high density polyethylene, or an acrylic material.

7. The shockwave head structure of claim 1, wherein the path regulation unit is a refracting unit positioned on the path of the energy wave.

8. The shockwave head structure of claim 7, wherein the shockwave waveguide body is filled with an acoustic medium.

9. The shockwave head structure of claim 7, wherein the refracting unit is made of a water sack, a high density polyethylene, or a silica gel material.
